(19) United States
(12) Patent Application Publication
(10) Pub. No.: US 2025/0018106 A1
(43) Pub. Date: Jan. 16, 2025

(54) PATCH PUMP

(71) Applicant: Unomedical A/S, Lejre (DK)

(72) Inventors: Omid Mostafaei, Lejre (DK); Lisa Jaunet van Kooten, Copenhagen (DK); Rune Byager, Frederiksberg (DK); Henrik Tipsmark, Zealand (DK)

(21) Appl. No.: 18/902,320

(22) Filed: Sep. 30, 2024

Related U.S. Application Data
(63) Continuation of application No. PCT/EP2023/059055, filed on Apr. 5, 2023.
(60) Provisional application No. 63/328,806, filed on Apr. 8, 2022.
(60) Provisional application No. 63/413,360, filed on Oct. 5, 2022.
(60) Provisional application No. 63/413,370, filed on Oct. 5, 2022.
(60) Provisional application No. 63/446,582, filed on Feb. 17, 2023.

(30) Foreign Application Priority Data
May 23, 2022 (GB) 2207515.4
Nov. 22, 2022 (GB) 2217425.4
Dec. 7, 2022 (GB) 2218352.9

(51) Int. Cl. A61M 5/142; A61M 5/165

(52) U.S. Cl. CPC A61M 5/14248; A61M 5/165; A61M 2205/75

(57) ABSTRACT

A patch pump for delivering a therapeutic agent to a patient in a subcutaneous manner, the patch pump comprising: a reservoir unit configured to store a therapeutic agent, a delivery unit having a cannula arranged to deliver the therapeutic agent to a patient in a subcutaneous manner, a pump unit arranged to pump the therapeutic agent along a fluid flow path extending from the reservoir unit to the cannula, and a filter secured in the fluid flow path and configured to filter the therapeutic agent prior to delivery to the patient.

The invention relates to a patch pump for subcutaneous infusion of a therapeutic agent into a patient and associated methods. Particularly, though not exclusively, the invention relates to patch pumps for subcutaneous infusion of insulin, heparin, apomorphine, arbidopa, or levodopa and/or levodopa products into a patient.

### BACKGROUND

For patients with diabetes, insulin therapy is often an important part of their treatment, helping to regulate blood sugar levels and store excess glucose for energy. There are two principal modes for delivering insulin. The first mode includes syringes and injector pens, which are used to inject a dose of insulin typically three to four times a day (depending on, inter alia, the type of diabetes and blood sugar levels of the patient). While these devices are simple and low cost, delivering each dose of insulin requires a needle stick. The second mode uses an infusion pump, sometimes called an insulin pump, which delivers controlled doses of insulin throughout the day. An infusion pump can be used to deliver insulin to a patient continuously (basal dose), on demand (bolus dose) or at scheduled intervals. Infusion pumps are more complex and expensive than syringes and pens, though enable improved regulation of blood sugar levels, for example by programmable delivery schedules, and requires fewer needle sticks.

The second mode is known as continuous subcutaneous insulin infusion (CSII) therapy. Infusion pump systems, such as patch pumps or infusion sets for CSII therapy may be worn by the patient. The systems typically include a combined infusion pump and reservoir fro containing an insulin drug, for example human insulin or analogue insulin, and a cannula (for example, a polymeric catheter or metal needle) for insertion subcutaneously into the patient. In the case of infusion sets, flexible tubing can connect the infusion pump to an infusion set worn by the patient. Once the cannula is inserted into the patient, it may remain in place for a period of time, i.e., days, to allow for continuous delivery of the insulin drug. The current recommended wear time for patch pumps is two to three days, to avoid problems that may arise relating to the patch pump itself or to the infusion site. However, such problems may still arise within recommended wear times, resulting in early removal of the patch pump and more frequent site rotation across infusion sites (for example buttocks, abdomen and arms).

While problems relating to the patch pumps have been well investigated and addressed in recent years, there remains little understanding and few solutions to address problems relating to the infusion site. Problems relating to the infusion site include pain, bleeding, infection, skin irritation, erythema, lipohypertrophy and lipoatrophy. Problems at the infusion site may lead to the build-up of scar tissue, which consequently lowers insulin sensitivity and increases the risk of hypoglycaemia, as well as having a cosmetic impact on patients. All these problems can deter patients from continuing to use their patch pumps, resulting in poorer patient outcomes.

It is known that problems at the infusion site are a consequence of the immune response to the presence of the cannula and the insulin drug in the body. The immune system responds by activating and progressing the foreign body reaction (FBR)—an inflammatory and fibrotic process that occurs upon introducing a foreign material into the body. In FBR, cells of the immune system identify foreign materials (such as unwanted biological, chemical, or physical species, present in infusible solutions) and attempt to degrade it, or otherwise encapsulate the material by forming a physical barrier to isolate it from the rest of the body. FBR is a problem for increasing wear times of patch pumps in CSII therapy, as the immune system reacts to the inserted cannula and the insulin drug. This limitation prevents realising the full potential of CSII therapy.

Generally, unwanted species, whether biological, chemical or physical, present in infusible solutions have undesirable consequences for patients.

It is an object of embodiments of the invention to provide an improved patch pump that attempts to circumvent FBR, increase wear times of infusion sets, and/or at least mitigate one or more problems associated with known arrangements.

### BRIEF SUMMARY OF THE DISCLOSURE

The invention is defined by the appended claims.

The present disclosure provides a patch pump for delivering a therapeutic agent to a patient in a subcutaneous manner, the patch pump comprising: a reservoir unit configured to store a therapeutic agent, a delivery unit having a cannula arranged to deliver the therapeutic agent to a patient in a subcutaneous manner, a pump unit arranged to pump the therapeutic agent along a fluid flow path extending from the reservoir unit to the cannula, and a filter secured in the fluid flow path and configured to filter the therapeutic agent prior to delivery to the patient.

In some examples, the filter is secured within the reservoir unit. In some examples, the filter is secured within a fluid port of the reservoir unit.

In some examples, the reservoir unit is arranged to receive the therapeutic agent from an external source. In some examples, the reservoir unit comprises a second fluid port for receiving the therapeutic agent from the external source. In some examples, the reservoir unit comprises a single fluid port for releasing the therapeutic agent.

In some examples, the filter is secured within the pump unit. In some examples, the battery unit comprises a controller arranged to pump the therapeutic agent along the fluid flow path. In some examples, the controller has a non-volatile memory having instructions stored thereon for pumping the therapeutic agent according to a pre-determined schedule. In some examples, the controller is configured to receive one or more instructions from an external device for operating the pump unit. In some examples, the battery unit comprises a power supply for powering the pump unit. In some examples, the patch pump comprises a dosing unit arranged to dispense the dose of the therapeutic agent to the pump unit. In some examples, the pump unit comprises the dosing unit.

In some examples, the patch pump comprises a filtration unit having an inlet end for receiving the therapeutic agent, and an outlet for discharging the therapeutic agent. In some examples, the filter is secured between the inlet of the filtration unit and the outlet of the filtration unit.

In some examples, the filtration unit is disposed upstream of the pump unit. In some cases, the filtration unit is disposed downstream of the reservoir unit. In some examples, the filtration unit is disposed downstream of the pump unit.

In some examples, the filtration unit comprises an inlet part for receiving the therapeutic agent at the inlet of the filtration unit. In some examples, at least a portion of the inlet part comprises a roughened inner surface for initiating bubble formation within the therapeutic agent when flowing over the roughened inner surface. In some cases, the inlet part comprises a downstream end having a flared profile arranged to direct the therapeutic agent towards the filter. In some examples, the inlet part is arranged to grip an end of a tubular element for fluidly connecting the delivery unit to the reservoir unit.

In some examples, the filtration unit is arranged to receive the therapeutic agent in a first direction, and wherein the filter is secured at an angle relative to the first direction. In some examples, the filter is secured at an acute angle relative to the first direction. In some examples, the filter is secured perpendicularly relative to the first direction. In some examples, the filter is secured in a parallel manner relative to the first direction.

In some examples, the filtration unit comprises a semi-permeable membrane in fluid communication with the fluid flow path and configured to vent gas contained with the fluid flow path.

In some examples, the filtration unit comprises an intermediate media secured in the fluid flow path, and the intermediate media is adapted to initiate bubble formation within the therapeutic agent as the therapeutic agent flows through the intermediate media. In some examples, the intermediate media is secured upstream of the filter. In some examples, the intermediate media comprises a porous section, and the fluid flow path passes through the porous section. In some examples, the intermediate media comprise an open-cell foam, for example a sponge-like material.

In some examples, the filter is secured within the delivery unit.

In some examples, the delivery unit comprises an inlet part arranged to receive the therapeutic agent. In some cases, the inlet part comprises a downstream end having a flared profile arranged to direct the therapeutic agent towards the filter. In some examples, the inlet part is arranged to grip an end of a tubular element for fluidly connecting the delivery unit to the reservoir unit.

In some examples, at least a portion of the inlet part comprises a roughened inner surface. In some examples, at least a portion of the downstream end of the inlet part comprises the roughened inner surface.

In some examples, the delivery unit comprises: an outlet end for receiving an upstream end of the cannula, and a flow redirecting part secured therein and arranged to fluidly connect the inlet part and the upstream end of the cannula. In some examples, the flow redirecting part is arranged to receive the therapeutic agent in a first direction and to discharge the therapeutic agent towards the upstream end of the cannula in a second direction different to the first direction. The first direction may be at an acute angle relative to the second direction. The first direction may be at 90 degrees relative to the second direction.

In some examples, the delivery unit comprises a semi-permeable membrane in fluid communication with the fluid flow path and configured to vent gas contained with the fluid flow path.

In some examples, the delivery unit comprises an intermediate media adapted to initiate bubble formation within the therapeutic agent. In some examples, the intermediate media is secured upstream of the filter. In some examples, the intermediate media comprises a porous section, and the fluid flow path is arranged to pass through the porous section. In some examples, the intermediate media is secured upstream of the filter. In some examples, the intermediate media comprises an open-cell foam, for example a sponge-like material.

In some examples, the patch pump comprises a magnetic filtration unit, preferably an electromagnetic filtration unit, arranged to apply a magnetic force to the therapeutic agent at one or more pre-determined positions along the fluid flow path to filter one or more charged elements from within the fluid flow path. In some examples, the electromagnetic filtration unit is powered by battery unit. In some examples, the pump unit can be arranged to charge one or more particles within the dose of the therapeutic agent. In some examples, a separate charging unit may be provided to charge one or more particles within the therapeutic agent., The charge applied may be a positive or negative charge to one or more particles within the dose of the therapeutic agent. In some cases, a porous intermediate media can be used as an electromagnetic filtration media and/or a bubble formation media. In some cases, the electromagnetic filtration unit is arranged to reduce one or more charged particles from within the therapeutic agent, for example magnesium and/or calcium to reduce the liquid hardness. In some examples the magnetic filtration unit comprises one or more permanent magnets arranged to apply the magnetic force. The permanent magnets advantageously filter out charged particles, such as iron.

There is also provided a patch pump for delivering a therapeutic agent to a patient in a subcutaneous manner, the patch pump comprising: a reservoir unit for storing a therapeutic agent, a delivery unit having a cannula arranged to deliver the therapeutic agent to a patient in a subcutaneous manner, a pump unit arranged to pump the therapeutic agent along a fluid flow path extending from the reservoir to the cannula, and a magnetic filtration unit, preferably an electromagnetic filtration unit, arranged to apply a magnetic force at one or more pre-determined positions along the fluid flow path so as to filter one or more charged elements from within the therapeutic agent prior to delivery to the patient.

In some examples, the magnetic filtration unit comprises an inlet for receiving the therapeutic agent and an outlet for discharging filtered therapeutic agent. In some cases, the magnetic filtration unit is external to the fluid flow path. In some cases, the magnetic force is applied across tubing containing the therapeutic agent.

In some examples, the electromagnetic filtration unit and the pump unit are connected to a common power supply. In some examples, the common power supply is contained within the pump unit.

### BRIEF DESCRIPTION OF THE DRAWINGS

Embodiments of the invention are further described hereinafter with reference to the accompanying drawings, in which:

FIG. 1 is an illustration of an exemplary patch pump housing;

FIG. 2 is a schematic representation of a first exemplary patch pump;

FIG. 3 is an illustration of an exemplary delivery unit;

FIG. 4 is a perspective cross-sectional view of a first exemplary delivery unit;

FIGS. 5A and 5B are cross-sectional views of a second exemplary delivery unit;

FIG. 6 is an illustration of an exemplary intermediate media;

FIGS. 7 and 8 are schematic representations of second and third exemplary patch pumps;

FIGS. 9A to 9C are illustrations of exemplary filtration units;

FIG. 10 is a schematic representation of a fourth exemplary patch pump;

FIG. 11 is an illustration of an exemplary electromagnetic filtration unit.

### DETAILED DESCRIPTION

The present description has particular application to patch pumps, such as patch pumps for delivering a therapeutic agent such as insulin, to a patient. However, other applications are contemplated, for example cannula access ports and infusion pump systems, such as insulin pumps for CSII therapy and infusion sets. The therapeutic agent may be an insulin drug, for example human insulin or analogue insulin. The presently described patch pumps are intended for delivering one ore more pre-determined doses of insulin to a patient at a single infusion site over an extended period of time. Used herein, an extended period of time is to be understood to mean at least four days. More specifically, an extended period of time may include four to seven days, seven or more days, seven to 10 days, and 10 or more days. An extended period of time may include 14 or more days. The patch pump may be used in closed loop systems, wherein the extended period of time may be at least equal to the wear time and/or lifetime of a CGM device.

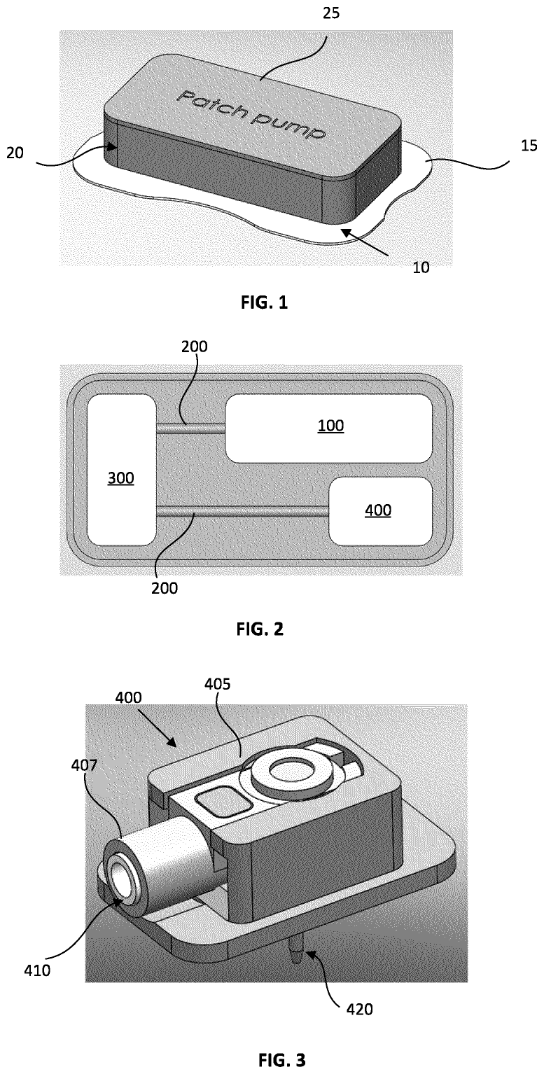

FIG. 1 is an illustration of an exemplary patch pump housing 10, including a plurality of sidewalls 20, a lid 25 and an adhesive patch 15 for affixing the patch pump housing 10 to the skin of a patient. The lid 25 and sidewalls 20 define a cavity within which the components necessary deliver a therapeutic agent 5 can be stored. It would be apparent the rectangular housing 10 is not essential and the housing 10 may have other profiles. Patch pumps typically have no external tubing and are a self-contained unit that allows a patient to administer and manage their treatment themselves. The patch pump housing 10 may be used with any of the patch pumps described below.

FIG. 2 is a schematic representation of a first exemplary patch pump and the lid 25 has been omitted for clarity. FIG. 2 shows a reservoir unit 100, a pump unit 300 and a delivery unit 400. Tubing 200 is used to connect the reservoir unit 100 to the pump unit 300, and to connect the pump unit 300 to the delivery unit 400.

The reservoir unit 100 contains the therapeutic agent (not shown) prior to delivery to the patient. The illustrated reservoir unit 100 includes only a single fluid port through which the therapeutic agent can be charged initially and discharged to the patient in a controlled manner using the pump unit 300. However, it would be apparent that this was not essential and that in some cases the reservoir unit 100 can contain multiple fluid ports, for example a dedicated inlet port for charging the reservoir unit 100 and a separate fluid port that can connect the reservoir unit 100 to tubing 200.

The pump unit 300 is used to pump a dose of the therapeutic agent from the reservoir unit 100 to the delivery unit 400 in a controlled manner to deliver a pre-defined dose of therapeutic agent 5. The pump unit 300 typically includes the battery and electronics necessary to control and power the electronic components within the patch pump. While the pump unit 300 is shown downstream of the reservoir unit 100 in FIG. 2, it would be apparent this was not essential, and in some cases the pump unit 300 can be arranged upstream of the reservoir unit 100. The pump unit 300 can also include a controller for communicating with an external device, for example that of the patient, so they can control or adjust the operation of the patch pump as required. The controller can be included as part of the pump unit 300, or may be a distinct control unit external to the pump unit 300. In some cases, a separate dosing unit (not shown) may be provided to dispense a pre-determined dose of the therapeutic agent to the pump unit 300 for delivery as described herein. In some cases, the pump unit 300 can both dispense the pre-determined dose and pump the predetermined dose of the therapeutic agent.

The delivery unit 400 receives the therapeutic agent 5 through tubing 200 and delivers the therapeutic agent 5 to the patient in a subcutaneous manner, typically through a cannula 420 which is secured to the delivery unit 400 as shown in FIG. 3.

FIG. 3 is an illustration of an exemplary delivery unit 400 which includes a base 405 to accommodate and secure the remaining parts of the delivery unit 400 in position within the patch pump housing 10. These include the cannula 420, a fluid part 407 through which the therapeutic agent 5 passes through to reach the cannula 420, and an inlet part 410 which holds the tubing 200 containing the therapeutic agent 5 in position and provides a fluid-tight seal around the tubing 200.

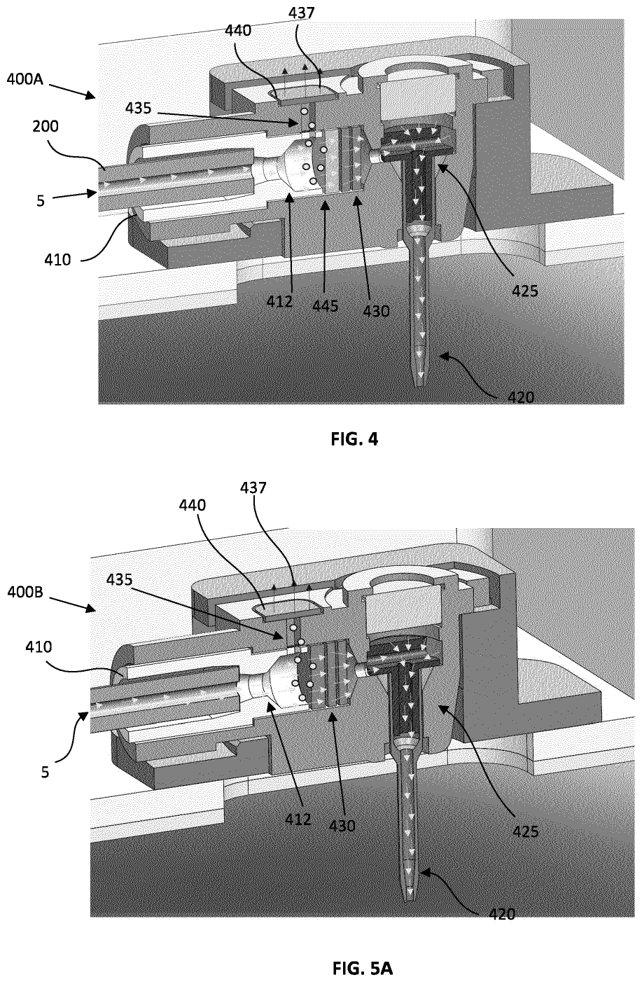

FIG. 4 is a perspective cross-sectional view of a first exemplary delivery unit 400A. The inlet part 410 includes an inlet for receiving the therapeutic agent 5 from the reservoir unit 100 (e.g. through tubing 200 from the pump unit 300) and an outlet through which the therapeutic agent 5 is directed towards a filter 430 secured within the fluid part 407. The filter 430 removes one or more unwanted species from within the therapeutic agent 5 before passing through a flow redirecting part 425 and into the cannula 420 for delivery to the patient. While the flow redirecting part 425 is shown directing the filtered therapeutic agent 5 into the cannula at an angle of approximately 90 degrees, it would be apparent this was not essential, and that the flow redirecting part 425 may be arranged differently to direct the fluid in any desired direction relative to the direction the filtered therapeutic agent is received. While a filter 430 is described, this can include one or more filter elements for filtering specific unwanted species from within the therapeutic agent 5. The inlet part 410 also secures the filter 430 in position within the fluid part 407. The inlet part 410 is shown held by a form-fitting connection, specifically inter-locking protrusions and recesses, but it would be apparent that the inlet part 410 may be secured to the fluid part 407 using any chemical or mechanical fixing means.

The inlet part 410 also secures a porous structure 445 upstream of the filter 430 within the fluid part 407. The porous structure 445 reduces the fluid pressure within the therapeutic agent 5 which causes dissolved gasses within the therapeutic agent 5 to form as the liquid passes through the porous structure 445 (see also FIG. 6). This advantageously results in the removal of dissolved gasses 437 contained within the therapeutic agent prior to delivery to the patient. It is preferable to remove bubbles 437 prior to delivery to reduce the risk of a foreign body reaction in the patient. The porous structure 445 initiates the formation of bubbles 437 which can pass through a vent 435, but not through the filter 430. A semi-permeable membrane 440 secured to the delivery unit 400 and in fluid communication with the fluid flow path via the vent 435 is used to vent bubbles from within the delivery unit 400. Advantageously, the pressures typically used to pump therapeutic agent 5 through along the fluid flow path are sufficient to force gas through the semi-permeable membrane 440. The semi-permeable membrane is preferably an air-permeable, liquid-impermeable membrane.

While a porous structure 445 is described herein, it would be apparent this was merely one example of an intermediate media suitable for initiating bubble formation in the therapeutic agent. By controlling the porosity of the porous structure 445 it is also possible to control the pressure of the therapeutic agent 5 as it passes through the delivery unit 400. One example of a porous structure 445 is shown in FIG. 6, where pores 449 are formed by a series of interconnected struts 447. The porosity of the porous structure 445 can be controlled in a number of ways, for example by altering the strut 447 thickness and/or the number of struts 447. The porous structure 445 may be followed by one or more layers of filter 430 to form a layered structure which restricts the ability of bubbles to pass through the filter 430 compared to if the gasses were dissolved in the therapeutic agent 5. The filter 430 can include one or more air-impermeable layers to prevent air from passing through the filter 430. While a porous structure 445 is described, the intermediate media can include other structures or functional materials to reduce the fluid pressure in the manner described. For example, the intermediate media may comprise any of a honeycomb structure, a mesh structure, a Zeolite or similar.

While it is desirable to remove dissolved gasses prior to filtering the therapeutic agent 5, it is possible to include one or more further units, such as vent units, to vent any bubbles formed in the therapeutic agent 5 prior to delivery to the patient. Such vent units may include a semi-permeable membrane fluidly connected to the fluid flow path as described herein.

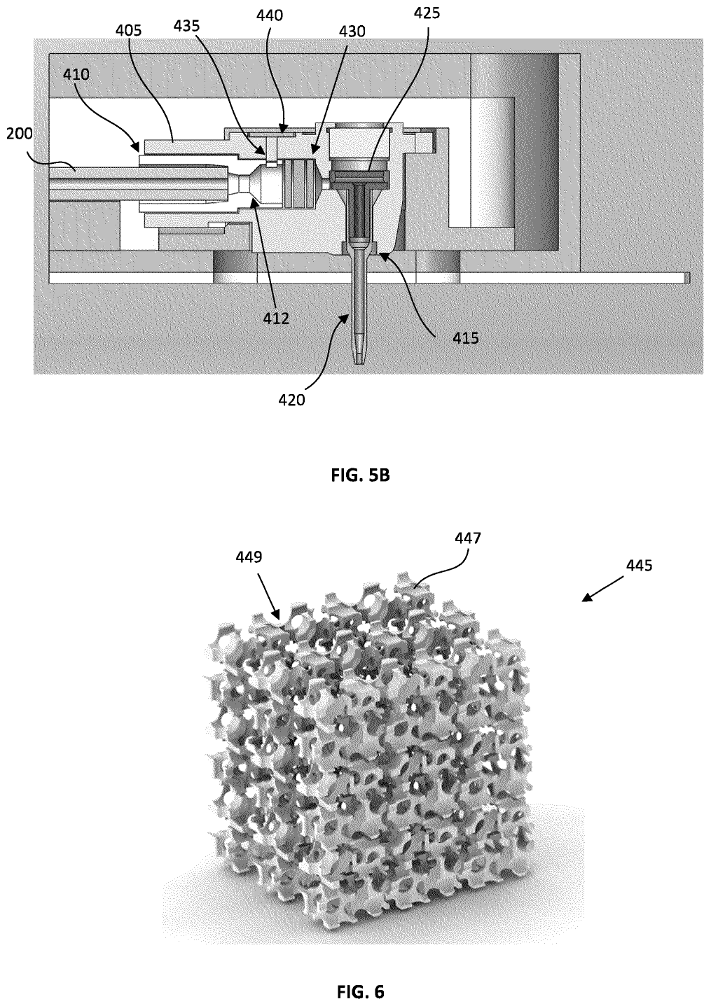

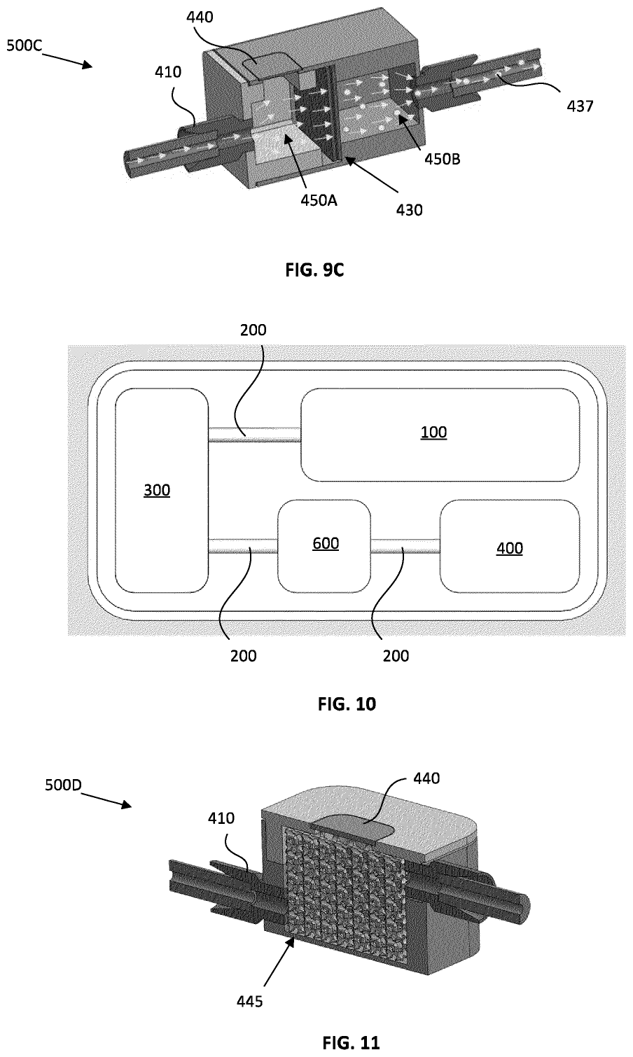

Independently of the porous structure 445, as the therapeutic agent 5 contacts and passes through the filter 430, this will induce a pressure drop across the filter 430. Upstream of the filter can be considered a high pressure region 450A, while downstream of the filter can be considered a low pressure region 450B (see FIGS. 9A-9C). It is in the low pressure region 450B that bubbles can form which can then be transported into the patient. It is therefore desirable to remove dissolved gasses from the therapeutic agent 5 prior to reaching the filter 430. Additionally or alternatively to providing a porous structure 445, the formation of bubbles can be achieved by lowering the pressure of the therapeutic agent 5 as it enters the delivery unit 400 by providing a flared opening 412 in the inlet part 410 (see FIGS. 4, 5A and, 5B). The flared opening 412 causes the fluid pressure of the therapeutic agent 5 to drop as it exits the inlet part 410 which initiates bubble formation. In the illustrated examples, the effect of the flared opening 412 is enhanced by including a narrowed section upstream of the flared opening 412.

Further additionally or alternatively to the porous structure 445 and/or the flared opening 412, it is possible to initiate bubble formation within the therapeutic agent 5 by passing the therapeutic agent 5 over a roughed surface. By increasing the surface roughness of the inlet part 410, this increases the number of nucleation sites on the inner surface of the inlet part 410, which in turn aids the formation of bubbles. It would be apparent that different surface roughness characteristics in the inlet part 410 can be used to control the level of bubble nucleation. A roughened surface can be achieved using mechanical abrasion of the inlet part 410 such that some or all of the inlet part 410 has a roughened surface. In some cases, a first portion of the inlet part 410 can be characterised by a first surface roughness parameter and a second portion of the inlet part 410 can have a second surface roughness parameter different to the first surface roughness parameter to indicate the second portion has a more rough surface finish than the first portion. The second portion of the inlet part 410 may be part of the flared opening 412 or be a separate downstream section from the inlet of the inlet part 410. Additionally or alternatively, the inlet part 410 may be provided with a plurality of raised surface protrusions which cause a turbulent flow as described above. Thus, while it is desirable to include a porous structure 445 to remove dissolved gasses, it is not essential. Other materials or structures can be used as an alternative or in combination with the porous structure to initiate the formation of bubbles within the therapeutic agent 5. The delivery units illustrated in FIGS. 4, 5A and 5B may be considered integrated delivery and filtration units as they include a filter 430.

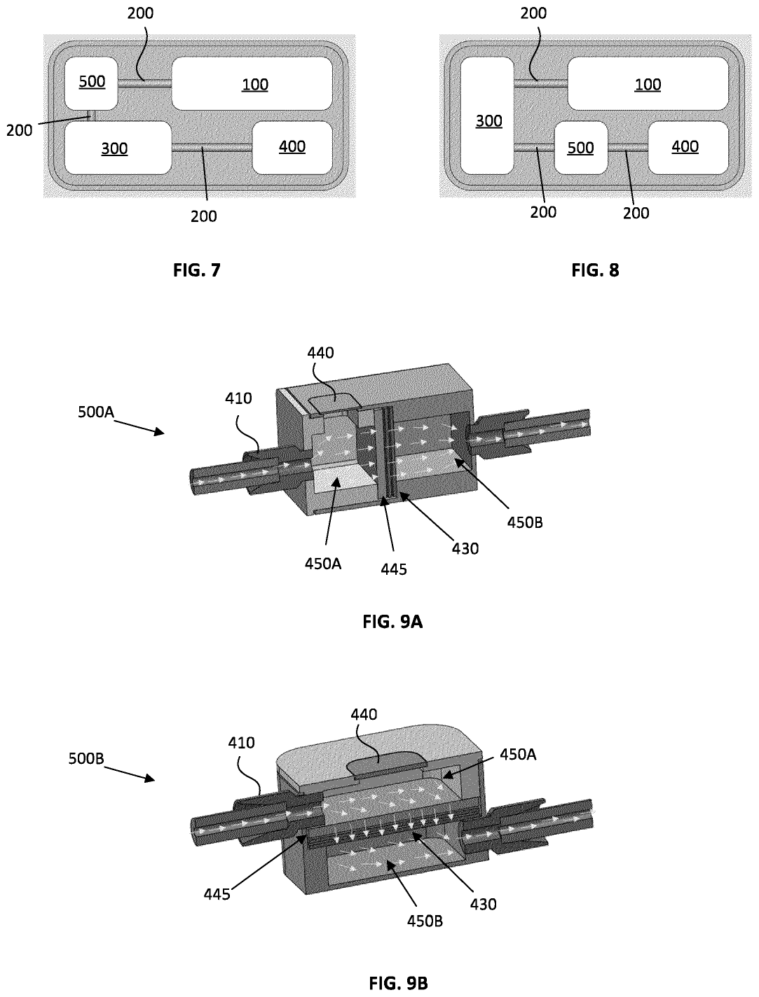

FIGS. 7 and 8 are schematic representations of second and third exemplary patch pumps, both of which include a dedicated filtration unit 500 to filter the therapeutic agent 5. It is advantageous to provide a dedicated filtration unit 500, as this unit can be designed to filter the therapeutic agent more efficiently than were the filter incorporated in another of the existing units of a patch pump. FIG. 7 shows the fluid flow path passing from the reservoir unit 100 to the filtration unit 500 and onto to the pump unit 300. FIG. 8 shows the fluid flow path passing from the pump unit 300 to the filtration unit 500 and onto to the delivery unit 400. It would be apparent the patch pump can include multiple filtration units 500 in addition to, or as an alternative to, an integrated delivery and filtration unit.

FIGS. 9A to 9C are illustrations of exemplary filtration units 500A-500C. These filtration units 500A-500C include an inlet part 410 for connecting tubing 200 to the filtration units and an outlet part for connecting the filtration unit 500A-500C to the downstream component (e.g. the pump unit 300 or the delivery unit 400). The inlet part 410 functions as described above in relation to the delivery unit 400 and will not be repeated here. The filtration units 500A-500C include a filter 430 and a porous structure 445 upstream of the filter 430 to facilitate the formation of bubbles within the therapeutic agent 5 prior to reaching the filter 430 as described above. The inlet part 410 connects the tubing 200 to an upstream chamber 450A of the filtration unit 500A-500C and any bubbles formed within the upstream chamber 450A are vented through the semi-permeable membrane 440 fluidly connected to the upstream chamber 450A. FIG. 9A shows the filter 430 and porous structure 445 arranged perpendicular to the direction of fluid flow from the inlet part 410 into the upstream chamber 450A. However, it is desirable to increase the surface area over which the filter 430 can act on the therapeutic agent 5, and so the filtration unit 500 can be arranged such that the filter 430 and the porous structure 445 are arranged perpendicular to the direction of fluid flow from the inlet part 410 into the upstream chamber 450A. It would be apparent that the filter 430 and/or porous structure 445 can be arranged independently of one another, and that the filter 430 and/or the porous structure 445 can be arranged an at acute angle relative to the direction of fluid flow from the inlet part 410 as required. An outlet part similar to the inlet part 410 is used to connect the filtration unit 500A-500C to the downstream tubing 200 to allow therapeutic agent 5 contained within the downstream chamber 450B to be discharged.

As the porous structure 445 is not essential it can be omitted. The filtration unit 500C of FIG. 9C does not include a porous structure 445, and as explained above, this would result in dissolved gasses forming bubbles in a downstream chamber 450B (i.e. downstream relative to the filter 430), as the filter 430 is unable to capture the bubbles 437. In this case, the bubbles within the therapeutic agent should be removed prior to the therapeutic agent reaching the patient to avoid causing a foreign body reaction. The removal of the bubbles can be achieved, for example, through a semi-permeable membrane 440 provided in the delivery unit 400 or in one or more vent units as explained above.

FIG. 10 is a schematic representation of a fourth exemplary patch pump that includes a reservoir unit 100, a pump unit 300, a delivery unit 400 and an electromagnetic filtration unit 600 for filtering charged particles within the therapeutic agent 5. The delivery unit 400 of FIG. 10 can be any of the delivery units described herein. In some cases, an electromagnetic filtration unit 600 may be the only filter within the patch pump. The electromagnetic filtration unit 600 has an electrical connection to the pump unit 300 so that the pump unit 300 and the electromagnetic filtration unit 600 share a common power supply which is the power supply of the pump unit 300. This is particularly advantageous as the size of the patch pump can be further reduced by not needing to provide a separate power supply for the electromagnetic filtration unit 600. In some cases, the electromagnetic filtration unit 600 can include a dedicated power supply independent of that used to power the pump unit 300. In FIG. 10, the electromagnetic filtration unit 600 is a separate module. However, it would be apparent that in some cases, the electromagnetic filtration unit can be embedded into other modules, for example those containing a filter 430, the delivery unit 400, the pump unit 300 or the reservoir unit 100.

FIG. 11 illustrates an exemplary electromagnetic filtration unit 600 containing a porous structure 445. The porous structure 445 can be charged to cause charged particles passing through the porous structure 445 to adhere to the porous structure and not be delivered to the patient. Particles, such as unwanted species, within the therapeutic agent can be charged with either a positive or a negative charge. Charging the particles within the therapeutic agent can be performed any point upstream of the electromagnetic filtration unit 600. By charging one or more particles within the therapeutic agent, for example by tagging any unwanted species with the charged particle, it is possible to filter any unwanted species as the therapeutic agent passes through the porous structure 445 in an efficient manner due to the greatly increased surface area compared to adhering the particles to the tubing 200. This effect is further enhanced by the non-linear fluid flow path through the electromagnetic filtration unit 600. While offsetting the inlet and outlet of the electromagnetic filtration unit 600 is one way of achieving the non-linear fluid flow path, it would be apparent this was not essential, and the porous structure 445 can be designed to direct the fluid flow in a predetermined manner. While a filter 430 is not included in the electromagnetic filtration unit 600, it would be apparent this could be included.

A separate battery unit can be provided to power the electromagnetic filtration unit 600, for example to charge one ore more unwanted species within the therapeutic agent and/or providing the electromagnetic force within the porous structure 445. The electromagnetic field created within the electromagnetic filtration unit 600 can have a static or a dynamic waveform. While a single controller can be used to control the different units described, it would be apparent this was not essential.

The electromagnetic filtration unit 600 may comprise one or more permanent magnets to filter out charged particles from within the therapeutic agent. One or more filtration units 500A-500C can be included in the patch pump of FIG. 10.

The patch pumps and filtration units 500A-500C described above each include a filter 430. The filter is intended to remove, for example by filtration, unwanted species present in the therapeutic agent to circumvent FBR. Used herein, unwanted species is to be understood to mean one or more species which may be present in the therapeutic agent, for example by design or accident, and which may be undesirable to remain in the therapeutic agent at the point of delivery to the infusion site. In particular, the filter may remove unwanted species that occur in insulin solutions. Such unwanted species may be particulate and/or molecular in nature. Examples of particulate unwanted species include plastic particles, dust and insulin agglomerates, which have been produced during manufacture, storage, sterilization, or handling of the infusion set and/or the insulin solution. Examples of molecular unwanted species include preservatives commonly used in insulin solutions, such as phenol, cresol (particularly m-cresol), benzyl alcohol, benzalkonium chloride, cetrimide, chlorobutanol, chlorhexidine, chlorocresol, hydroxy benzoates, phenethyl alcohol, phenoxyethanol and phenylmercuric nitrate.

The filter may include any filter material capable of removing one or more unwanted 5 species from the therapeutic agent. To remove particulate unwanted species, the filter material may provide a physical filter medium for removing unwanted species by size exclusion, including whereby the filter material functions as a molecular sieve. Additionally, or alternatively, to remove molecular unwanted species, the filter material may provide a chemical filter medium for removing unwanted species by sorption, for example by adsorption or ion exchange, whereby the filter material binds with the molecular unwanted species to retain them within the filter. The filter material has a plurality of passageways, for example pores (i.e., interconnected hollow voids), extending therethrough to allow fluid flow through the filter.

The filter may be a modular filter including first and second sub-filters arranged to allow fluid flow therethrough in series for progressively removing different unwanted species from the therapeutic agent, for example unwanted species of varying sizes and/or varying molecular composition. Accordingly, the first sub-filter may include a filter material different to that of the second sub-filter. The filter may similarly include a third, a fourth and so on sub-filters.

Suitable filter materials include foams made of a cellulose, a polyurethane, a polyester, a polyether, a collagen or the like. The foam includes a plurality of passageways in the form of interconnected pores extending therethrough to allow fluid flow through the filter. The foam may be any foam capable of removing particulate unwanted species from an insulin solution. The foam may remove particulate unwanted species from an insulin solution by a size exclusion process. While a foam is described herein, it would be apparent other structures, such as a membrane or a sheet or similar may be used in place of a foam and the properties described in relation to foams apply equally to membranes or sheets.

Particularly suitable filter materials are disclosed in earlier patent applications, including U.S. Pat. Nos. 4,083,906 and 11,197,949, the contents of which are incorporated herein by reference.

Suitable filter materials include an ion-exchange resin, including functionalised porous or gel polymers, which may remove unwanted species from an insulin solution by a gel permeation chromatography process. Moreover, gel polymers may be used to coat passageways in the filter material.

Generally, the filter material may be selected to have at least one material property that may facilitate the infusion of insulin at a single infusion site over an extended period of time, and thereby increase wear times, for example at least four days, including four to seven days, seven or more days, seven to 10 days, 10 or more days, and 14 or more days.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A patch pump for delivering a therapeutic agent to a patient in a subcutaneous manner, the patch pump comprising:
a reservoir unit configured to store a therapeutic agent,
a delivery unit having a cannula arranged to deliver the therapeutic agent to a patient in a subcutaneous manner,
a pump unit arranged to pump the therapeutic agent along a fluid flow path extending from the reservoir unit to the cannula, and
a filter secured in the fluid flow path and configured to filter the therapeutic agent prior to delivery to the patient.

2. The patch pump according to claim 1, wherein the filter is secured within the reservoir unit.

3. The patch pump according to claim 1 or 2, wherein the reservoir unit is arranged to receive the therapeutic agent from an external source.

4. The patch pump according to any preceding claim, wherein the filter is secured within the pump unit.

5. The patch pump according to any preceding claim further comprising a filtration unit having an inlet for receiving the therapeutic agent, and an outlet for discharging the therapeutic agent, and wherein the filter is secured between the inlet of the filtration unit and the outlet of the filtration unit.

6. The patch pump according to claim 5, wherein the filtration unit is disposed upstream of the pump unit.

7. The patch pump according to claim 5, wherein the filtration unit is disposed downstream of the pump unit.

8. The patch pump according to any of claims 5 to 7, wherein the filtration unit comprises an inlet part for receiving the therapeutic agent at the inlet of the filtration unit, and wherein at least a portion of the inlet part comprises a roughened inner surface for initiating bubble formation within the therapeutic agent when flowing over the roughened inner surface.

9. The patch pump according to any of claims 5 to 8, wherein the filtration unit is arranged to receive the therapeutic agent in a first direction, and wherein the filter is secured at an angle relative to the first direction.

10. The patch pump according to any of claims 5 to 9, wherein the filtration unit comprises a semi-permeable membrane in fluid communication with the fluid flow path and configured to vent gas contained with the fluid flow path.

11. The patch pump according to any of claims 5 to 10, wherein the filtration unit comprises an intermediate media secured in the fluid flow path, and wherein the intermediate media is adapted to initiate bubble formation within the therapeutic agent as the therapeutic agent flows through the intermediate media.

12. The patch pump according to any preceding claim, wherein the filter is secured within the delivery unit.

13. The patch pump according to claim 12, wherein the delivery unit comprises an inlet part arranged to receive the therapeutic agent, and wherein the inlet part comprises a downstream end having a flared profile arranged to direct the therapeutic agent towards the filter.

14. The patch pump according to claim 13, wherein at least a portion of the inlet part comprises a roughened inner surface.

15. The patch pump according to any of claims 12 to 14, wherein the delivery unit comprises:
an outlet end for receiving an upstream end of the cannula, and a flow redirecting part secured therein and arranged to fluidly connect the inlet part and the upstream end of the cannula,
wherein the flow redirecting part is arranged to receive the therapeutic agent in a first direction and to discharge the therapeutic agent towards the upstream end of the cannula in a second direction different to the first direction.

16. The patch pump according to any of claims 12 to 15, wherein the delivery unit comprises a semi-permeable membrane in fluid communication with the fluid flow path and configured to vent gas contained with the fluid flow path.

17. The patch pump according to any of claims 12 to 16, wherein the delivery unit comprises an intermediate media adapted to initiate bubble formation within the therapeutic agent.

18. The patch pump according to any preceding claim comprising an electromagnetic filtration unit arranged to apply a magnetic force to the therapeutic agent at one or more pre-determined positions along the fluid flow path to filter one or more charged elements from within the fluid flow path.

19. The patch pump according to claim 18, wherein the electromagnetic filtration unit and the pump unit are connected to a common power supply.

20. The patch pump according to claim 19, wherein the common power supply is contained within the pump unit.

21. A patch pump for delivering a therapeutic agent to a patient in a subcutaneous manner, the patch pump comprising:
a reservoir unit for storing a therapeutic agent,
a delivery unit having a cannula arranged to deliver the therapeutic agent to a patient in a subcutaneous manner,
a pump unit arranged to pump the therapeutic agent along a fluid flow path extending from the reservoir to the cannula, and
a magnetic filtration unit, arranged to apply a magnetic force at one or more predetermined positions along the fluid flow path so as to filter one or more charged elements from within the therapeutic agent prior to delivery to the patient.

22. The patch pump according to claim 21, wherein magnetic filtration unit is an electromagnetic filtration unit.

23. The patch pump according to claim 21 or 22, wherein the magnetic filtration unit comprises an inlet for receiving the therapeutic agent and an outlet for discharging filtered therapeutic agent.

24. The patch pump according to any of claims 21-23, wherein the magnetic filtration unit is external to the fluid flow path.

25. The patch pump according to any of claims 21-24, wherein the magnetic force is applied across tubing containing the therapeutic agent.

26. The patch pump according to claim 22, wherein the electromagnetic filtration unit and the pump unit are connected to a common power supply.

27. The patch pump according to claim 26, wherein the common power supply is contained within the pump unit.
